(19) United States
(12) Patent Application Publication
(10) Pub. No.: US 2003/0219356 A1
(43) Pub. Date: Nov. 27, 2003

(54) Virus eliminator

(76) Inventor: Richard J. Cavanaugh, Las Vegas, NV (US)

Correspondence Address: Richard J. Cavanaugh, 6818 Jeanette St., Las Vegas, NV, 89131, US

(21) Appl. No.: 10/055,676

(22) Filed: May 22, 2002

(51) Int. Cl. A61M 1/14; A61M 37/00

(52) U.S. Cl. 422/44; 604/6.08

(57) ABSTRACT

The enclosed application titled Virus Eliminator is described physically and functionally. This permits the elimination of viruses in the blood stream. The scope of the invention has many ramifications of being used in a clinical practice of medicine, including private physicians, hospitals and clinics.

### CROSS-REFERENCE TO RELATED APPLICATIONS

Not applicable

### BACKGROUND—FIELD OF INVENTION

This invention relates to human blood, when exposed to ultraviolet light, can be purged of many unwanted cells that cause diseases.

### BACKGROUND—DESCRIPTION OF PRIOR ART

Photo chemotherapy with psoralen-containing plan extracts was employed in Egypt and India in 1500 B.C. for the treatment of vitiligo. El Mofty at the University of Cairo first used a purified psoralen for the treatment of vitiligo in 1947. In 1974, Parrish et al. reported successful treatment of severe psoriasis with 8-methoxypsoralen (P) and UVA, and coined the acronym PUVA. PUVA has been approved for the treatment of vitiligo and psoriasis. Its widespread use with extensive follow-up has provided comprehensive data on toxicity and efficacy.

Currently a process known has helix developed by Cerus Corporation has apparently developed this apparatus which uses ultraviolet light and renders killer viruses bacteria harmless. This process will be used in conjunction with the Red Cross and blood centers to screen pathogens and viruses in donated blood for blood replacement in the human body. This method has been developed to treat large quantities of blood where the end result will be a product that is free of contamination.

Presently a number of companies are in different stages of developing kidney dialysis machines and/or virus inactivation devices. These machines will be used in hospitals and blood centers. They are expensive to operate. They require approximately six hours to radiate the blood of an individual which is complicated procedure, time consuming and expensive.

This particular medical device would be portable and inexpensive to own or to purchase and/or operate. It is designed to render a treatment rather than perfect a product.

### SUMMARY

This device is capable of eliminating a number of viruses from human blood through the exposure of ultraviolet light as described in the diagrams.

### Objects and Advantages

Accordingly, in addition to the advantages already described, the following summary is included:

(a) to provide a treatment on an individual basis for individuals who have various contaminations including viruses.

(b) Provides a very rapid and economical method.

(c) It is mobile and easily operated.

(d) Relatively inexpensive to operate.

(e) It takes relative a shorter period of time for a decontamination of blood.

(f) It offers the individual recipient an opportunity to reduce his state of contagion.

Still further objects and advantages will become apparent from a consideration of the ensuing description in the claim and the drawings.

### DRAWING FIGURES

FIG. 1, No. 100 shows lateral view of entire unit including electrical outlet, quartz tube ultraviolet light.

FIG. 2, No. 100 shows opposite view from above.

FIG. 3, No. 100 shows end view including electrical outlet quartz tubing and ultraviolet light.

FIG. 4, No. 100 shows opposite end.

FIG. 5 shows quartz tubing enlarged with serrations cut into tube.

FIG. 6 shows outlet on magnified scale of quartz tubing.

FIG. 7 shows quartz tube on lateral view with enlarged serrations.

FIG. 8 shows lateral view from the opposite side.

FIG. 9 shows sagital section of quartz tubing showing inside diameter of tube.

FIG. 10, No. 100 shows quartz tubing in function position, electrical outlet, electrical connections for ultraviolet tube and base, which holds tube.

FIG. 11 shows holder for ultraviolet light.

FIG. 12 shows holder for ultraviolet light including screws for supporting base.

FIG. 13 shows supporting stand for ultraviolet light.

FIG. 14 shows lateral view of supporting stand for quartz tube.

FIG. 15 shows lateral view of supporting base for ultraviolet light. Two views: one end view and one oblique.

### REFERENCE NUMERALS IN DRAWINGS

A list of Reference Numerals is included

### DESCRIPTION OF FIGURES

Every reference numeral in the specification is on the drawings.

### Method of Operation

Helical Quartz Blood Treatment Coil

Utilizing “indents” impressed into quartz tubing angled 45° to axis of blood flow provides full exposure of human blood cells to ultraviolet light due to turbulence caused by the indents.

Human blood, when exposed to ultraviolet light, can be purged of many unwanted cells that cause diseases. Therefore it is the purpose of this invention to provide maximum exposure of ultraviolet light to human blood cells.

Quartz is a mandatory material for the tubing, as ultraviolet light will pass through quartz unimpeded.

When blood is passed through a straight quartz tube, some cells are protected by the shadow of other cells and are not eradiated by the ultraviolet light.

Straight tubing of a given length equates to exposure time to the ultraviolet light but due to that length, the tubing is long and cumbersome for an easy-to-use device that is convenient.

Bending the quartz tubing into a helical (or spiral) shape reduces the size of the apparatus without reducing the physical length of the tubing.

Drawings depict a three-coil unit but any number of coils can be added to vary the exposure time to eradiate cells that otherwise would not be neutralized in a short time span.

Elimination of the “shadow” effect within the tubing is achieved by the inclusion of “indents” in the coil. These indents are impressed into the quartz tubing while the quartz tubing is in a soft state during the forming of the coil shape.

These indents are impressed at a forty-five degree (45°) angle relative to the direction of flow. These indents cause the blood to spiral within the quartz tubing providing a “mixing” effect allowing full exposure of all cells to the ultraviolet light.

### Component Identification for Each Figure

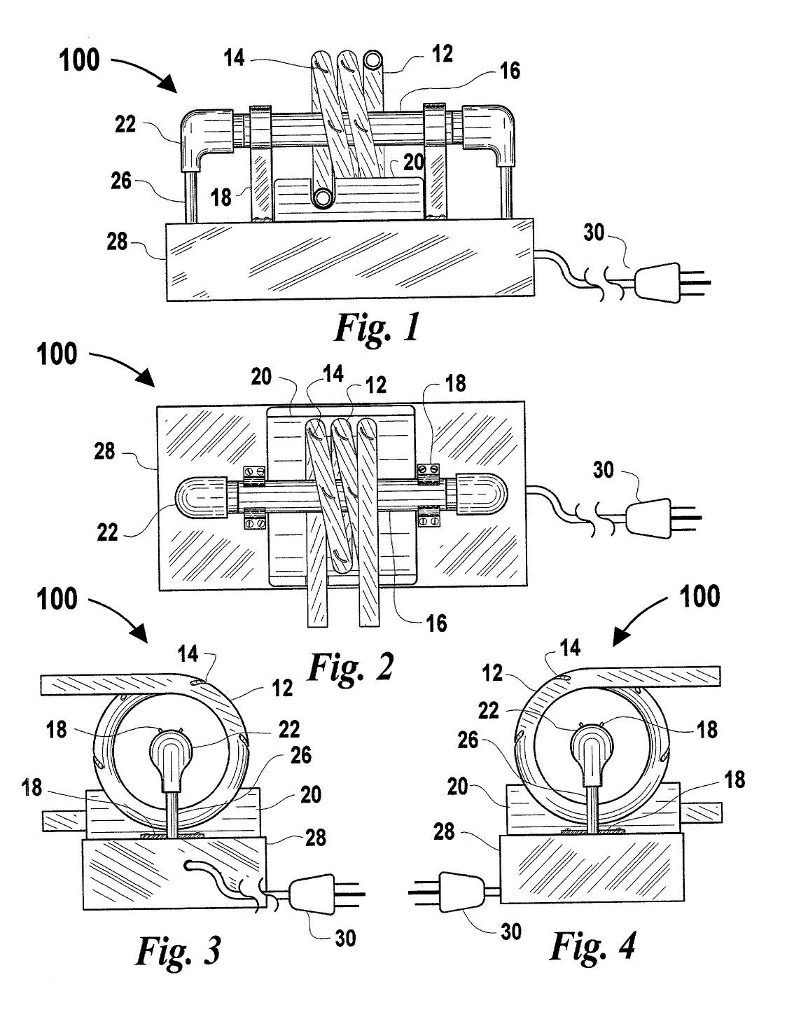

FIG. 1 (Front view)

Number

100 . . . Blood treatment unit complete.

12 . . . Quartz tubing

14 . . . Indent (6 ea, in each coil) 3 coils.

16 . . . Ultraviolet lamp.

18 . . . Spring clip lamp (16) support.

20 . . . Soft rubber support for quartz coil (12).

22 . . . Rubber cap protecting lamp (16) electrical contacts (24).

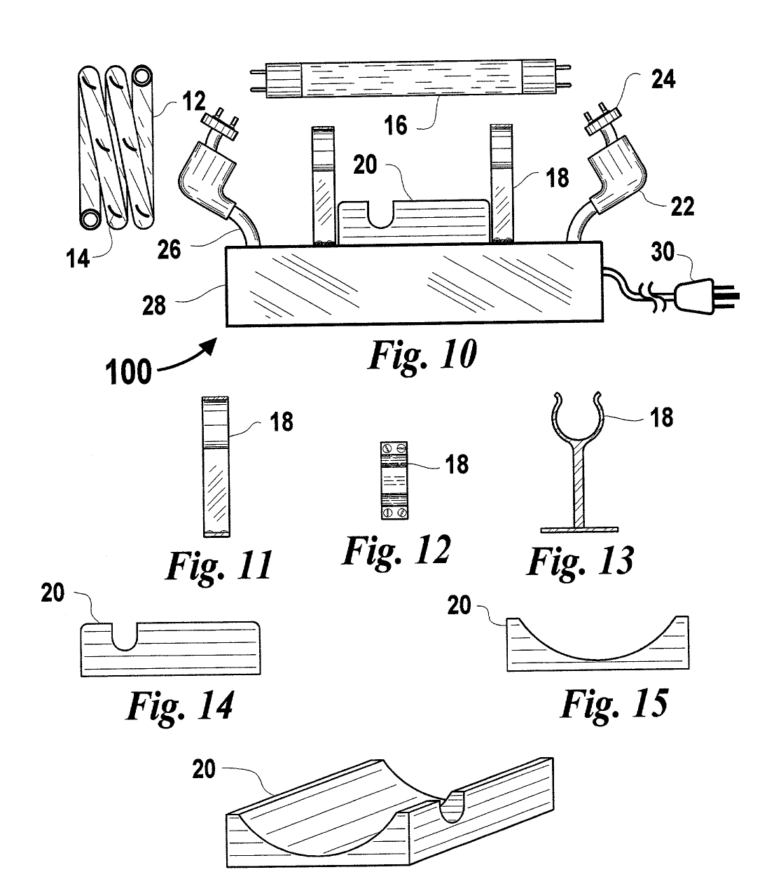

24 . . . Not shown in FIG. 1 (See FIG. 10).

26 . . . Electric supply wire for lamp (16).

28 . . . Plastic base (or other material) to house electric components for lamp (16).

30 . . . Electrical plug to power lamp (16).

FIG. 2 (Top view)

All numbers are same as FIG. 1 except item (24) & (26) not shown in this view.

FIG. 3 (Right end view)

All numbers are same as FIG. 1 except item (24) not shown in this view.

FIG. 3 (Right end view)

All numbers are same as FIG. 1 except item (24) not shown in this view.

FIG. 4 (Left end view)

All numbers are same as FIG. 1 except item (24) not shown in this figure.

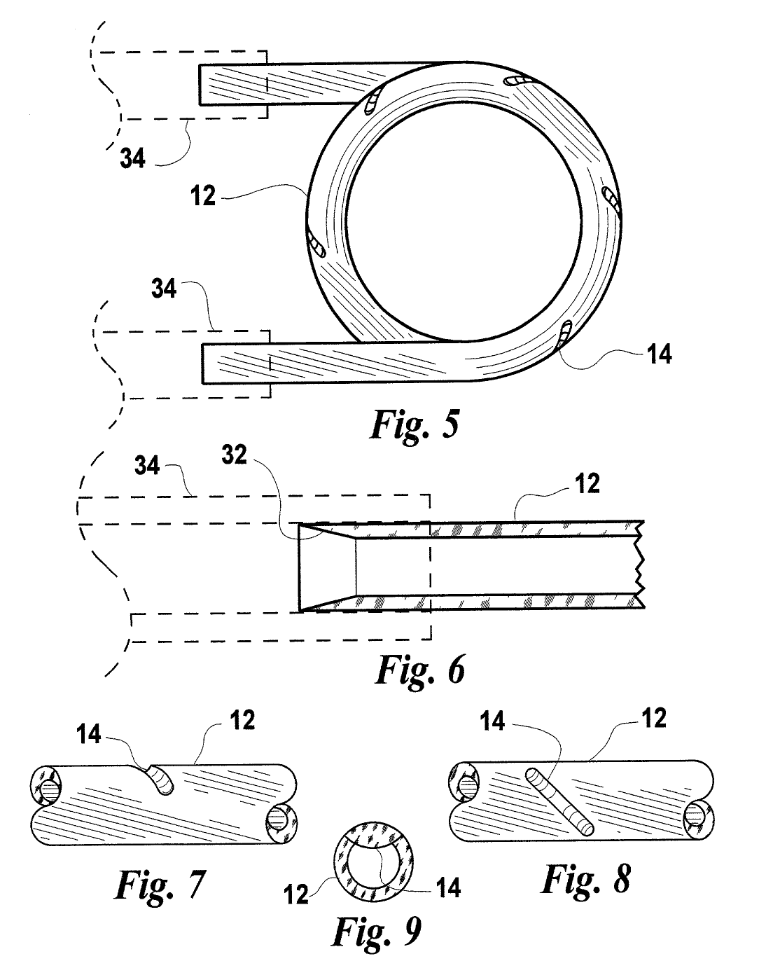

FIG. 5 (Right side view)

12 . . . Quartz Coil.

14 . . . Indents.

34 . . . Medical tubing to and from source.

FIG. 6 Cross section of end (in/out)

32 . . . End of Quartz tubing inside diameter reduced to sharp end to preclude restriction of fluid when entering or exiting coil.

34 . . . Medical tubing.

FIG. 7 (Side view of indent)

12 . . . Section of Quartz tubing.

14 . . . Indent in Quartz tubing.

FIG. 8 (Top view of indent)

12 . . . Section of Quartz tubing.

14 . . . Indent in Quartz tubing (top view).

FIG. 9 (End view of Quartz tubing)

12 . . . Cross section of Quartz tubing to show inside of tubing at juncture of indent.

14 . . . Indent from inside Quartz tubing.

FIG. 10 (Exploded view of 100)

All numbers same as FIG. 1 but adding item (24).

24 . . . Connection wire to ultraviolet lamp.

FIG. 11 (Side view)

18 . . . Lamp spring clip support.

FIG. 12 (Top view)

18 . . . Lamp spring clip support.

FIG. 13 (End view)

18 . . . Lamp spring clip support.

FIG. 14 (End view)

20 . . . Rubber mount pad for Quartz tubing.

FIG. 15 (Side view)

20 . . . Rubber mount pad for Quartz tubing.

FIG. 16 (Isometric view)

20 . . . Rubber mount pad for Quartz tubing.

1. The entire part consists of a piece of quartz tubing bent on itself in a circular fashion having an entrance and an exit for the flow of blood and containing a number of small indentations on the internal lumen of the tubing. This structure and design assures the greatest amount of exposure of the blood within the tube to the effect of ulta violet light
